(19) United States
(12) Patent Application Publication
(10) Pub. No.: US 2023/0139535 A1
(43) Pub. Date: May 4, 2023

(54) SYSTEMS AND METHODS OF SIMPLE AND AUTOMATABLE PROTEIN DIGESTION USING MAGNETIC BEADS

(71) Applicant: Phenomenex, Inc., Torrance, CA (US)

(72) Inventors: Yongzheng Cong, Rancho Palos Verdes, CA (US); Avelino C. Sanchez, Torrance, CA (US); Emmet Welch, Folsom, CA (US); Jianli Zhao, Cypress, CA (US)

(21) Appl. No.: 18/047,799

(22) Filed: Oct. 19, 2022

Related U.S. Application Data
(60) Provisional application No. 63/257,417, filed on Oct. 19, 2021.

(51) Int. Cl. G01N 33/543; C12N 9/50

(52) U.S. Cl. CPC G01N 33/54326; C12N 9/50; G01N 2446/20

(57) ABSTRACT

The disclosure provides methods and kits for preparing a protein analyte for characterization. The method comprises pretreating a protein analyte in the presence of a magnetic bead in a changing magnetic field; and enzymatically digesting the protein analyte with an endopeptidase enzyme to provide a plurality of peptides for analysis by, for example, LC-MS/MS. The process can be performed in a single tube without the need for desalting or buffer exchange, in less than 2 hours.

### CROSS REFERENCE TO RELATED APPLICATION

This application claims the benefit of and priority to U.S. Provisional Application Ser. No. 63/257,417, filed 19 Oct. 2021, the disclosure of which is incorporated by reference herein in its entirety.

### REFERENCE TO A SEQUENCE LISTING

The instant application contains a Sequence Listing, which has been submitted electronically in XML format and is hereby incorporated by reference in its entirety. Said XML file, created on Oct. 18, 2022, is named Sequence-Listing-18777-0004USU1.xml and is 4,533 bytes in size.

### BACKGROUND

Protein digestion is a labor-intensive sample preparation process used for proteomic analysis. Protein digestion may be employed to aid identity confirmation of a protein therapeutic and or to monitor degradative events such as oxidation or deamidation. Peptide mapping may be used for sequence verification of a purified protein with a known amino acid sequence. Peptide mapping analysis comprises enzymatic protein digestion, peptide separation, and peptide detection. Protein digestion may include reduction and alkylation of cysteine thiols, and enzymatic cleavage of the protein into peptides, followed by LC-MS/MS analysis. Traditional methods of protein digestion can lead to low reproducibility, high artifacts, and incompatibility with automation.

Enzymatic digestion can cause artificial modification of the peptides due to exposure to various buffers, reducing and alkylating agents, and elevated temperatures. Protein modifications such as asparagine deamidation, N-terminal glutamine cyclization, and methionine oxidation may be promoted by elevated temperatures or high pH which may be exacerbated over time. The amount of these artificial modifications or artifacts is directly proportional to the incubation time of protein samples in the reduction/alkylation buffer and in the digestion buffer, where the peptides are completely solvent exposed.

There is a need for a simple and automatable protein digestion method/system that improves reproducibility, throughput, and reduces artifacts for proteomic analysis.

### SUMMARY OF THE INVENTION

The disclosure provides improved methods, systems and kits for protein digestion, comprising magnetic beads-aided protein pre-treatment using electromagnetic mixing, and magnetic beads enabled protein digestion, using magnetic beads that respond to a changing magnetic field. The whole workflow can be conducted on a single device without moving the vials/plate from start to finish. The whole workflow is simple and easy to automate. In contrast, traditional workflows involve movement of vial/plate, centrifugation, and extra consumables, which is complex and difficult to automate.

A simple and automatable workflow process is provided, and is enabled by a novel magnetic mixing technology, with which liquid can be efficiently mixed by high response magnetic beads that travel through the liquid at high speed driven by a changing magnetic field. This mixing technology can be used for both protein pre-treatment and digestion at one spot/location, which simplifies the workflow and is compatible with automation. In preferred embodiments, the denaturant is compatible with mass spectrometry to avoid desalting by centrifugation.

The disclosure provides a method of preparing a protein analyte for characterization comprising pretreating a protein analyte in the presence of first magnetic beads in a changing magnetic field to provide a pretreated protein analyte; and enzymatically digesting the pretreated protein analyte to provide a plurality of peptides. The characterization may comprise analyzing the plurality of peptides comprising LC-MS/MS.

The pretreating may comprise mixing the protein analyte and a chaotropic agent in an aqueous buffer with the first magnetic beads in the changing magnetic field to provide a denatured protein solution. The pretreating may comprise adding a reducing agent to the denatured protein solution to provide a reduced, denatured protein analyte. The pretreating may comprise exposing the denatured protein analyte to an alkylating agent to provide an alkylated protein analyte. The pretreating may comprise quenching excess alkylating agent by adding a reducing agent to the alkylated protein analyte to provide the pretreated protein analyte.

The digesting may comprise blending the pretreated protein analyte with an endopeptidase enzyme in the presence of the first magnetic beads in a changing magnetic field to provide the plurality of peptides.

The endopeptidase enzyme may be selected from the group consisting of a trypsin, chymotrypsin, LysC, LysN, AspN, GluC, and ArgC. The endopeptidase enzyme may be a free endopeptidase enzyme or an immobilized endopeptidase enzyme. The immobilized endopeptidase enzyme may be immobilized on the surface of second magnetic beads.

The first magnetic beads and or second magnetic beads independently may comprise a ferrimagnetic particle, superparamagnetic particle, ferromagnetic particle, paramagnetic particle, or mixtures thereof. The first magnetic beads and or second magnetic beads independently may have a high magnetic response having a Bmax in a range of from about 20 emu/g to about 250 emu/g, 40 emu/g to 200 emu/g, 50 emu/g to 150 emu/g, or 80 emu/g to 100 emu/g. The first magnetic beads and or second magnetic beads independently may have a high surface area of >m²/g,>7 m²/g, or >10 m²/g,

The first magnetic beads and or second magnetic beads independently may further comprise a coating, optionally wherein the coating is selected from the group consisting of a silica shield, silane linker, and a polymer coating.

The first magnetic beads and or second magnetic beads independently may comprise a functional group-coated surface, optionally wherein the functional-group coated surface is selected from the group consisting of carboxyl groups, amino groups, hydroxyl groups, thiol groups, tosyl groups, epoxy groups, alkyl groups, vinyl groups, and aryl groups.

The first magnetic beads and or second magnetic beads independently may comprise a bioaffinity adsorbent, optionally selected from the group consisting of streptavidin, avidine, neutravidin, captavidin, and biotin.

In some embodiments, the first magnetic beads do not comprise an immobilized endopeptidase enzyme.

The changing magnetic field may be generated in an electromagnetic mixer. The electromagnetic mixer may comprise a plurality of electromagnets capable of generating an AC driven oscillating magnetic field.

The protein analyte may be selected from the group consisting of a monoclonal antibody, an isolated immunoglobulin, a recombinant protein, an isolated protein, and a complex protein sample; optionally, wherein the complex protein sample is selected from the group consisting of a tissue sample, blood sample, serum sample, and plasma sample.

The chaotropic agent may be an organic chaotropic agent. The organic chaotropic agent may be selected from the group consisting of trifluoroethanol (TFE), ethanol, 2-propanol, n-butanol, and phenol. The chaotropic agent may be an immobilized chaotropic agent; optionally wherein the chaotropic agent is immobilized on the first magnetic beads or on third magnetic beads.

The reducing agent may be selected from the group consisting of dithiothreitol (DTT), dithioerythritol (DTE), tris(2-carboxyethyl) phosphine hydrochloride (TCEP), beta-mercaptoethanol (βME), and L-glutathione (GSH).

The alkylating agent may be selected from the group consisting of 2-iodoacetamide (IAM), iodoacetic acid (IAC), and chloroacetamide (CAA).

The pretreating may be completed in a period of time within a range of from 5 to 90 minutes, or 10 to 60 minutes. The enzymatically digesting may be completed in a period of time from 5 to 60 minutes, or from 10 to 60 minutes. In some embodiments, the pretreating and the enzymatically digesting may be performed sequentially or simultaneously.

A kit is provided for preparing a protein analyte for characterization comprising a first container comprising first magnetic beads; a chaotropic agent; a reducing agent; an alkylating agent; and an endopeptidase enzyme. The first magnetic beads may comprise a functional group-coated surface, optionally wherein the functional-group coated surface is selected from the group consisting of carboxyl groups, amino groups, hydroxyl groups, thiol groups, tosyl groups, epoxy groups, alkyl groups, vinyl groups, and aryl groups; optionally wherein the first magnetic beads do not comprise an immobilized endopeptidase enzyme. The endopeptidase enzyme may be selected from the group consisting of a trypsin, chymotrypsin, LysC, LysN, AspN, GluC, and ArgC. The endopeptidase enzyme may be a free endopeptidase enzyme or an immobilized endopeptidase enzyme. The immobilized endopeptidase enzyme may be immobilized on second magnetic beads. The chaotropic agent may be a free chaotropic agent or an immobilized chaotropic agent. The immobilized chaotropic agent may be immobilized on the first magnetic beads or on third magnetic beads.

A system is provided comprising a kit according to the disclosure, and an electromagnetic mixer comprising a plurality of electromagnets capable of generating an AC driven oscillating magnetic field.

### BRIEF DESCRIPTION OF THE DRAWINGS

FIG. 1 shows a transmission electron microscopy (TEM) image of eMagBeads according to the disclosure. The exemplary beads are protected by 10 nm silica.

FIG. 2 shows comparative workflow schemes for tryptic digestion of a protein analyte. The prior art “in solution” workflow (upper panel) requires several manual operations including moving the vial between steps, the need for a desalting step, the need for a filtration step, and the need for a centrifugation step. The prior art in solution workflow is also difficult to automate and requires greater than 2 hours to complete workflow. In contrast to the prior art in solution workflow, the inventive eMag tryptic digestion workflows (lower panel) reduce the number of manual steps, because there is no need to move the vial between steps, no need for desalting, no need for filtration, and no need for centrifugation. The inventive tryptic digestion methods are easy to automate and require less than 2 hours to complete workflow.

FIG. 3 shows ultraviolet (UV) spectroscopy (upper panel) and high-resolution mass spectrometry results (MS)(lower panel) for three different tryptic digests of Herceptin including prior art in solution digestion, inventive eMag-Immobilized trypsin, and inventive eMag-free trypsin. FIG. 3 (lower panel) shows LC-MS data in Table 1 including number of unique peptides, number (#) missed cleavages, % trypsin artifacts, VSNK (SEQ ID NO: 3) deamidation area %, VVSVLTVLHQDWLNGK (SEQ ID NO: 1) deamidation area %, GFYPSDIAVEWESNGQPENNYK peptide (SEQ ID NO: 2) deamidation area %, DTLMISR (SEQ ID NO: 4) oxidation area %, autovalidated sequence coverage (%), and total area of validated peptides. Both of the inventive eMag workflows result in at least comparable tryptic digest results when compared to prior art “in solution” workflow, as indicated by the LC-MS data.

### DETAILED DESCRIPTION OF THE INVENTION

The present disclosure provides improved and simplified methods and kits for endopeptidase digestion of protein analytes, for example, for characterization by LC-MS/MS analysis. The methods may be performed without the need for moving vials, filtration, centrifugation, or desalting, and are amenable to automation.

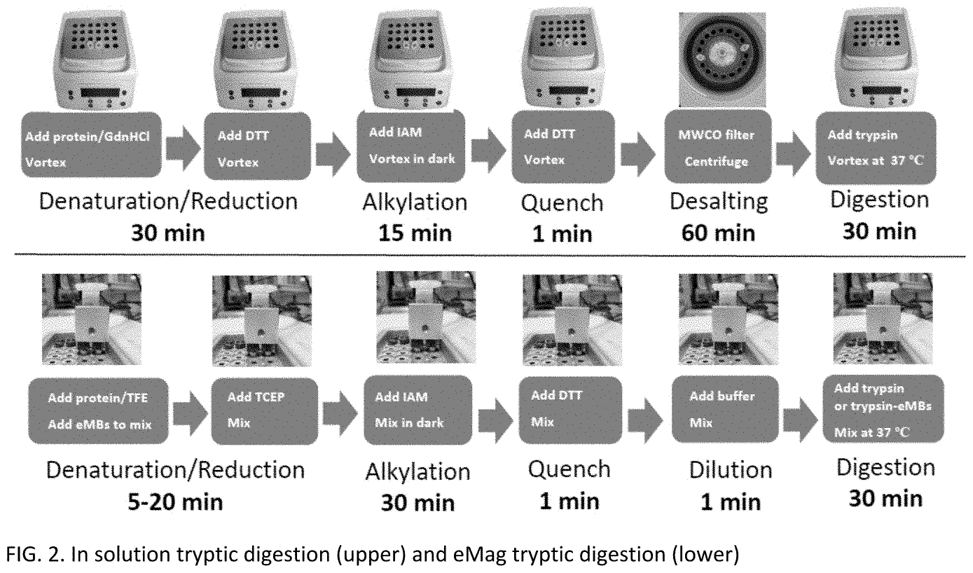

Prior art “in solution” tryptic digestion workflow of a protein analyte typically includes denaturation and reduction with vortex mixing (30 min), alkylation (15 min), quench (1 min), desalting with centrifugation (60 min), and digestion with free trypsin with vortex at 37° C. (30 min) prior to LC-MS/MS characterization of tryptic digest, as illustrated in FIG. 2 (upper panel). See, for example, Da Ren et al., 2009, An improved trypsin digestion method minimizes digestion-induced modifications on proteins, Anal Biochem 392 (1):12-21. The prior art “in solution” workflow requires several manual operations including moving the vial between steps, the need for a desalting step, the need for a filtration step, and the need for a centrifugation step. In consequence, the in solution tryptic digestion is also difficult to automate and requires greater than 2 hours to complete workflow.

Improved methods and kits for tryptic digestion workflow of a protein analyte are provided herein. Representative inventive eMag tryptic digestion workflow scheme is illustrated in FIG. 2 (lower panel): denaturation and reduction employ efficient mixing with electromagnetic beads in a changing magnetic field (carboxylated eMBs), alkylation (30 min), quench (1 min), dilution (1 min) and digestion using either trypsin-immobilized eMBs (Ty-eMBs) or free trypsin in a changing magnetic field (30 min). In contrast to the prior art in solution workflow, the inventive eMag tryptic digestion workflows (lower panel) reduce the number of manual steps, do not require moving the vial between steps, do not require desalting, do not require filtration, do not require centrifugation, are easy to automate, and require less than 2 hours to complete workflow.

### Definitions

The terminology used herein is for the purpose of describing particular embodiments only and is not intended to be limiting of the disclosure.

The singular forms “a”, “an” and “the” are intended to include the plural forms as well, unless the context clearly indicates otherwise.

The term “and/or” or “and or” refers to and encompasses any and all possible combinations of one or more of the associated listed items.

The term “about,” when referring to a measurable value such as an amount of a compound, dose, time, temperature, and the like, is meant to encompass variations of 10%, 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

The term “ferrimagnetic particles” refers to particles comprising a ferrimagnetic material. Ferrimagnetic particles can respond to an external magnetic field (e.g., a changing magnetic field), but can demagnetize when the external magnetic field is removed. Thus, the ferrimagnetic particles may be efficiently mixed through a sample by external magnetic fields as well as efficiently separated from a sample using a magnet or electromagnet, but can remain suspended without magnetically induced aggregation occurring.

The term “remanence” refers to residual magnetism that a material retains after a magnetic field has been removed. Materials that have a high remanence after the magnetic field has been removed retain a large magnetic field strength, whereas materials that have a low remanence after the magnetic field has been removed have a small magnetic field strength or zero magnetic field strength. The remanence of the magnetic materials may be in a range of fro about 0 emu/g to about 30 emu/g, about 0 emu/g to about 20 emu/g, about 1 emu/g to about 10 emu/g, about 3 emu/g to about 5 emu/g, or less than, equal to, or greater than about 0 emu/g, 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30 emu/g.

As used herein, the term “functional group-coated surface” refers to a surface which is coated with moieties which each have a free functional group which is bound to the magnetic particle; as a result, the surfaces of the magnetic particles are coated with the functional group containing moieties. The functional group may be employed to covalently attach a bioaffinity absorbent for biological molecules in solution. In one example, the functional group is a carboxylic acid. A suitable moiety with a free carboxylic acid functional group is a succinic acid moiety in which one of the carboxylic acid groups is bonded to the amine of amino silanes through an amide bond and the second carboxylic, acid is a free carboxylic acid group attached or tethered to the surface of the magnetic particle. The functional group-coated surface may be selected from carboxyl groups, amino groups, hydroxyl groups, thiol groups, tosyl groups, epoxy groups, alkyl groups, vinyl groups, or aryl groups.

The functional groups may be utilized to covalently attach a “bioaffinity adsorbent” such as a streptavidin, avidine, neutravidin, captavidin, biotin, and the like.

According to further examples the surface can be covalently bound to an enzyme, protein, deoxyribonucleic acid (DNA), ribonucleic acid (RNA), or an immunoglobulin. The enzyme may be an endopeptidase enzyme. The enzyme may be a trypsin enzyme.

The term “peptide sequence coverage” (SQ %) may be used as a measure for both the completeness of the protein digestion and the detection efficiency of the various tryptic peptides and is a common way in proteomics to define the digestion rate (Hustoft et al. (2012). A Critical Review of Trypsin Digestion for LC-MS Based Proteomics, Integrative Proteomics, Hon-Chiu Leung (Ed.), ISBN: 978-953-51-0070-6, InTech, Available from: www.intechopen.com/books/integrative-proteomics/a-critical-review-of-trypsin-digestion-for-lc-ms-basedproteomics).

The term “alkylating efficiency” is defined as the quantitative relation between the number of detected cysteine residues that have been modified by the alkylating agent during the reaction and the total number of cysteine residues detected in the sample, as determined by analyzing the sample using mass spectrometry, optionally followed by a database search. One non-limiting example includes alkylation of cysteine with an alkylating agent such as chloroacetamide (CAA) or iodacetic acid (IAC) that increases the mass of the peptide by 57.021 or 58.005 Daltons, respectively. This increase in mass is measured using a mass spectrometer and quantified during the database search.

Magnetic Particles

Any appropriate magnetic particles may be employed, so long as they can be mixed throughout a container. Magnetic particles suitable for mixing in response to changing magnetic fields are described in WO 2020/018919, Beckman Coulter, Inc.

Sample processing methods and systems for are provided for mixing, separating, filtering, or otherwise processing a sample (e.g., a fluid sample) by utilizing magnetic particles that are caused to move under the influence of a magnetic assembly disposed about the periphery of a container containing the sample. The magnetic beads may comprise a ferrimagnetic particle, superparamagnetic particle, ferromagnetic particle, paramagnetic particle, or mixtures thereof.

The magnetic particles may be ferrimagnetic particles. The ferrimagnetic particles may be efficiently manipulated (e.g., moved) by a changing magnetic field generated by a magnetic assembly. The ferrimagnetic particles can have a high response to magnetic fields, such that the ferrimagnetic particles are easily mixed into a sample when in the presence of an external changing magnetic field. The ferrimagnetic particles can also have a low residual magnetism, such that the ferrimagnetic particles are not magnetically attracted to one another when an external changing magnetic field is removed. As a result, the ferrimagnetic particles can remain suspended without magnetically induced aggregation occurring after mixing and thus do not inhibit binding or elution.

Further, the magnetic particles should remain suspended in the sample for a suitable time after mixing. One of skill will recognize that a number properties of the magnetic particles will affect this property. For example, the density, as well as the remanence (e.g., residual magnetism), of the magnetic particles can influence the length of time of suspension in the sample after the changing magnetic field is removed. In some examples it is desirable to separate the magnetic particles from the sample. In these examples, the magnetic particles can be magnetically separated from the container using a collection component, such as a magnet or an electromagnet, as described herein.

The magnetic particles employed in the present disclosure are sufficiently responsive to magnetic fields such that they can be efficiently moved through a sample. In general, the range of the field intensity could be the same range as any electromagnet as long as it is able to move the particles. For example, the magnetic field has an intensity of between about 1 OmT and about 100 mT, between about 20 mT and about 80 mT, and between about 30 mT and about 50 mT. In some examples, more powerful electromagnets can be used to mix less responsive microparticles. In some examples, the magnetic field can be focused into the sample as much as possible. Also, the electromagnets can be as close to the sample as possible since the strength of the magnetic field decreases as the square of the distance.

The ferrimagnetic particle may comprise a ferrite. A ferrite includes a ceramic material that comprise an oxide of iron in combination with inorganic compounds of metal, non-metal, or metalloid atoms. For example, a ferrite can comprise iron(III) oxide (Fe₂O₃) blended with one or more additional metallic elements, such as barium, manganese, nickel, zinc; titanium, or any other suitable metallic element. Other examples of ferrites include Fe₂TiO₂, FeTiO₂, MnFeO₄, NiFe₂O₄, MgFe₂O₄. further examples of ferrites include an iron core including a sulfide or an oxyhydroxide such as FCTSS, Fe₃S₄, FeS, or FeOOH.

Magnetite (Fe₃O₄) is an example of a magnetic material useful in the examples described herein that is an example of a ferrite. Magnetite contains both Fe²⁺ and Fe³⁺ ions. In some cases, the electron spins of the Fe²⁺ and Fe³⁺ ions can be coupled in a crystalline structure such that the magnetite is ferrimagnetic, as described herein. However, in some examples, ferrimagnetic particles comprise any ferrimagnetic material (e.g., ferrite). According to some examples, the ferrimagnetic material (e.g., ferrite) may not be magnetite (Fe₃O₄), however in some examples, magnetite is a suitable ferrimagnetic material.

Ferrites can be categorized into two main families (hard ferrite and soft ferrites) based on their magnetic coercivity (e.g., the material's ability to withstand an external magnetic field without becoming demagnetized).

Hard ferrites have a high magnetic coercivity as well as a high remanence after magnetization. Hard ferrites can be used to make permanent magnets, as hard ferrites do not demagnetize easily in the absence of an external magnetic field, as they can have a high remanence. Examples of hard ferrites include strontium ferrite and barium ferrite.

Soft ferrites have a low magnetic coercivity. Soft ferrites also have a low remanence after magnetization. The magnetization of soft ferrites is easier to change than hard ferrites. Further, the magnetization of soft ferrites can easily reverse direction without dissipating large amounts of energy (e.g., via hysteresis losses). Soft ferrites can also have a high electrical resistivity, thus preventing the formation of eddy currents in the material, which is another source of energy loss.

Soft ferrites can include manganese-zinc (MnZn) ferrite and nickel-zinc (NiZn) ferrite. Thus, in some examples the ferrimagnetic particles comprise MnZn ferrite. In other examples, the ferrimagnetic particles comprise NiZn ferrite. Ferrimagnetic particles comprising MnZn ferrite and/or NiZn ferrite can become magnetized in the presence of an external magnetic field, and thus are able to be moved in the presence of the external magnetic field, but do not aggregate due to magnetically induced aggregation after the external magnetic field is removed, since they have a low remanence.

Some ferrites can be considered to be semi-hard ferrites. Semi-hard ferrites have properties that are between the properties of soft ferrites and the properties of hard ferrites. For example, cobalt ferrite (CoFe₂O₄) is a semi-hard ferrite, which can be magnetized in the presence of an external magnetic field (e.g., a changing magnetic field generated by a magnetic assembly), but does not have a high remanence after the external magnetic field is removed, such that the ferrimagnetic particles comprising a cobalt ferrite core do not aggregate due to magnetically induced aggregation.

The magnetic particles can be a variety of shapes, which can be regular or irregular. In some examples, the shape maximizes the surface areas of the particles. For example, the magnetic particles can be spherical, bar shaped, elliptical, or any other suitable shape. The magnetic particles can be a variety of densities, which can be determined by the composition of the core. In some examples, the density of the magnetic particles can be adjusted with a coating, as described herein.

The strength of the magnetic field may be determined in Gauss. The gauss is the unit of magnetic flux density B in the system of Gaussian units and is equal to Mx/cm2 or or g/Bi/s². Gauss is a unit used to measure the strength of a magnetic field and a gaussmeter instrument may be used to make that measurement. The higher the number of Gauss, the more force the magnetic field will have, so the greater the distance will be reached from the surrounding magnet.

The magnetic particle may comprise a magnetic material having a maximum magnetic field strength (Bmax) in a range of from about 20 emu/g to about 250 emu/g, 40 emu/g to 200 emu/g, 50 emu/g to 150 emu/g, or about 80 emu/g to 100 emu/g. The Bmax may be >40, >50, >60, or >70 emu/g. The Bmax may be measured by SQUID (superconducting quantum interference device). The magnetic particle or magnetic bead may have a super high magnetic response, for example, having a Bmax of in a range of about 80-100 emu/g, or about 89 emu/g. This is compared to many commercially available magnetic beads having <40 emu/g.

In some embodiments, the magnetic particle may comprise a magnetic material having a maximum magnetic field strength (Bmax) in a range of from about 20 emu/g to about 250 emu/g and a remanence in a range of from about 0 emu/g to about 30 emu/g.

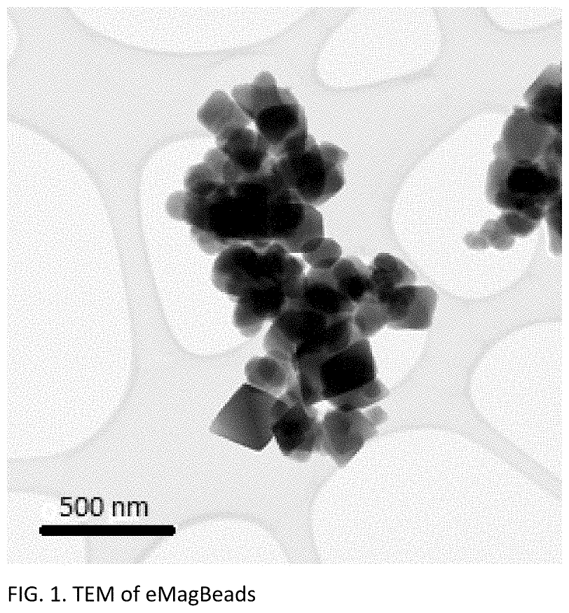

The magnetic particle may range from about 1 nm mean diameter to about 1 mm mean diameter. In some examples, the magnetic particles may have a mean diameter in a range of 50 nm to 500 nm. In some examples, the magnetic particles may have a mean diameter in a range of 100 to 200 nm. The magnetic bead may have a diameter in a range of about 80 to about 120 nm, or about 100 nm. The mean diameter may be measured by microscopy, for example, by TEM imaging, as shown in FIG. 1.

The magnetic particles can be substantially solid or can have some degree of porosity. Where the magnetic particles do include some degree of porosity, a pore size of the individual pores can be in a range of from about 5 A to about 1000 A, about 50 A to about 500 A. At least a plurality of the pores can be through pores (e.g., extending fully between opposed surfaces). The pore sizes or total porosity of the magnetic particles can be determined according to many suitable methods. For example, the bulk volume of an ideal (e.g., non-porous) magnetic particle can be determined and then the volume of the actual porous skeletal material can be determined. The porosity is then calculated by subtracting the volume of the actual porous skeletal material from the ideal magnetic particle. The porosity of the magnetic particle or individual pore size can also be determined through optical measurements using a microscope and processing the images to measure the individual pores.

The magnetic particles have sufficient surface area to permit efficient binding of an endopeptidase. In some examples, a surface area of the magnetic particles can be in a range of from about 0.1 m²/g to about 500 m²/g, about 1 m²/g to about 200 m²/g, or about 10 m²/g to about 100 m²/g. In some embodiments, the magnetic particles or magnetic beads have surface area>5 m²/g, >7 m²/g, or >10 m²/g. The surface area may be measured by BET surface analysis. (Brunauer-Emmett-Teller (BET) surface area analysis provides specific surface area evaluation of materials by nitrogen multilayer adsorption measured as a function of relative pressure using a fully automated analyzer. The technique encompasses external area and pore area evaluations to determine the total specific surface area in m²/g.

The magnetic particles described herein can include several different materials. To the extent that mixtures of materials are present, the total magnetic content of the magnetic particles can constitute at least 50 wt % of the magnetic particle, at least 70 wt % of the magnetic particle, at least 80 wt % of the magnetic particle, at least 90 wt % of the magnetic particle, or even 100 wt % of the magnetic particle.

The magnetic particles can include any of those described herein. The non-magnetic material constituting the balance of the magnetic particles can include any of the coating materials described herein, for example. Non-magnetic material can be used as a coating to encapsulate the magnetic portion of the magnetic particle, they can also be used as a functional component to interact with and bind an analyte of interest. Non-magnetic material can also act as filler component.

The magnetic particle or magnetic bead may be surface functionalized with a carboxyl, amino, hydroxyl, silica, streptavidin, or endopeptidase enzyme moiety. The magnetite particle or magnetic bead may have a magnetite core. The magnetite particle or magnetic bead may have a magnetite core coated with a silica shield layer. The silica shield layer may be attached to a silane linker. The silane linker may be attached to a polymer. The polymer may be surface functionalized with a carboxyl, amino, hydroxyl, silica, streptavidin surface functionality. In a specific embodiment, the magnetic bead or magnetic particle has a carboxyl surface functionality. The magnetic bead or magnetic particle may be covalently bound to an endopeptidase enzyme. The magnetic bead or magnetic particle may be covalently bound to a trypsin.

The magnetic particle may be coated and/or functionalized by any method known in the art. The coating can be, for example, a polymer layer, or a silica layer. Example polymer layers can include polyethylene, polystyrene, poly methyl methacrylate, polyvinyl alcohol, or any other suitable polymer.

A single layer or multiple layers of coating may be employed. For example, magnetic particle may be coated with a silica layer, a polymer coating, and/or functional group.

For example, synthesis of core-shell Fe₃O₄ nanoparticles (NPs) may be performed via hydrolysis of tetraethyl orthosilicate (TEOS) in the presence of Fe₃O₄ nanoparticles to provide silica-coated magnetite core-shell particles. For example, Fe₃O₄ NPs may be dispersed in water using an ultrasonic water bath, then mixing with aq. ammonia solution. (25 wt %) and ethanol. TEOS may be added dropwise into Fe₃O₄ suspension with stirring at room temperature overnight. the product may be separated using an external magnet, washed with water and dried at 50° C. The particles may be characterized by x-ray diffraction (XRD), transmission electron microscopy (TEM), high-resolution TEM, selected area electron diffraction (SAED), and UV-Vis absorption spectra (UV-Vis). Hui et al., 2011, Nanoscale, 3(2):701-5. See also WO2020018919, which is incorporated herein by reference in its entirety.

The silica-coated magnetite core-shell particles may be treated with 3-aminopropyltriethoxysilane (silane KH-550) to provide amino functionalized silica-coated magnetic beads. The amino functionalized silica-coated magnetic beads may be treated with ClCH₂CO₂Na to provide carboxyl functionalized magnetic beads.

A polymer coated magnetic core may be prepared by, for example, dispersing ˜4 g of 100 nm magnetite core in 100 ml water under stirring. 10 mL of acrylic acid is added to the flask under stirring with K2S₂O₈ to get a uniform suspension. The suspension is heated to 80° C. and stirred for 15 hrs. The suspension is cooled to room temperature and a permanent magnet is used to collect the solids from the suspension. The collected solids are washed with water and dried at 60° C. to provide a poly(acrylic acid) coated magnetic bead.

One example of a type of magnetic bead (eMB) developed in the present disclosure includes a magnetite core surrounded from the inner to outer direction by a silica shield, a silane linker, and a polymer. The polymer may be covalently attached to a surface functionality such as a carboxyl, amino, hydroxyl, or silica group. The surface functionality may be used for covalent attachment of, for example, a bioaffinity adsorbent such as streptavidin or an endopeptidase enzyme such as trypsin. The eMB may exhibit a high magnetic response, for example, having a Bmax of about 89 emu/g, as compared to <40 emu/g for commercial magnetic beads. The eMB may exhibit a high surface area of >10 m²/g.

The trypsin (e.g., bovine trypsin, recombinant porcine trypsin, TPCK-treated trypsin), or chaotropic agent, may be immobilized to the surface of the magnetic particle using a surface functionality by any appropriate method known in the art.

In one example, trypsin immobilized magnetic beads may be produced by mixing 0.5 g poly(acrylic acid) coated magnetic beads according to the disclosure with 20 mL 0.1 M sodium phosphate buffer (pH 7.5), and 50 mg TPCK-treated trypsin in a flask. The mixture is stirred to prepare a uniform suspension. 200 mg of 1-cyclohexyl-3-(2-morpholinoethyl)carbodiimide metho-p-toluenesulfonate is added to the suspension. The suspension is kept at 4° C. for 24 hrs under stirring. The product is collected using a permanent magnet and washed with water for 5 times. The product is then re-dispersed in 50 mM acetic acid in water and stored at 4° C.

In another example, trypsin immobilized magnetic beads may be produced by treating carboxylated magnetic beads according to the disclosure with EDC (1-ethyl-3-[3-dimethylaminopropyl]carbodiimide hydrochloride) activation chemistry to produce an active ester intermediate, followed by exposure to the endopeptidase enzyme (e.g., trypsin) wherein a primary amine such as a lysine side chain may be covalently attached via the active ester to form a covalently attached immobilized endopeptidase enzyme. Specifically, carboxylated eMagbeads may be activated using 1-ethyl-3-[3-dimethylaminopropyl]carbodiimide hydrochloride (EDC) chemistry. The beads may be incubated for 1 h at room temperature with a solution of 26 mg EDC (Sigma, St. Louis, Mo.) per gram of beads dissolved in 0.1 M 2-(N-morpholino)ethanesulfonic acid (MES) buffer, pH 4.5. Activation may be terminated by rinsing several times with MES buffer and DI water. To complete the immobilization, the activated beads may be incubated with a 40 μg endopeptidase enzyme per gram of beads of 0.1 M sodium phosphate buffer at pH=7.0. The process may be completed by blocking remaining active sites using a solution of phosphate buffered saline (PBS) and 0.1% v/v Tween 20. Following immobilization of the enzyme, beads may be washed several times with water or 1.0 MNaCl. The enzyme-immobilized beads may be preserved at 4-8° C. in a solution of deionized water.

In some examples, the trypsin coated magnetic beads or surface-functionalized magnetic beads may be purchased commercially. For example, magnetic particles with immobilized trypsin and streptavidin may be purchased from Perfinity (West Lafayette, Ind.). Silanol coated beads may be obtained from Chemicell (Berlin, Germany), and carboxyl functionalized superparamagnetic beads may be obtained from Interchim (Montlucon, France).

Reagents

A chaotropic agent may be used to denature or partially denature the protein analyte in solution (denaturant). The “chaotroptic agent” is a compound that can disrupt the hydrogen bonding network in water solution between water molecules and protein analytes in the solution, and may be used to denature or partially denature the protein analyte. Chaotropic agents increase the entropy of the system by interfering with intermolecular interactions mediated by non-covalent forces, e.g., hydrogen bonds, Van der Waals forces, and hydrophobic effects. Any appropriate chaotropic agent may be employed as a denaturant. The chaotropic agent may be an organic alcohol or a chaotropic salt. The organic alcohol may be a C₂₋₆ organic alcohol, wherein the C₂₋₆ may be a straight or branched-chain alkyl or aryl group, optionally comprising one or more halo substituents. The optional halo substituents may be F, Cl, Br, I. The optional halo substituents may be F or Cl. Examples of organic alcohol chaotropic agents include trifluoroethanol (TFE), ethanol, 2-propanol, n-butanol, and phenol. Examples of other chaotropic agents may include urea or thiourea. Examples of chaotropic salts may include guanidine hydrochloride (Gdn HCl), guanidinium thiocyanate, magnesium chloride, sodium chloride, sodium bromide, lithium perchlorate, lithium acetate, sodium acetate, potassium acetate, NaClO₄, NaNO₃, CF₃COONa, CCl₃COONa, and NaSCN. The chaotropic agent may be purchased commercially from, for example, Sigma-Aldrich. Preferably, the chaotropic agent is mass spectrometry (MS)-compatible so that no desalting is needed. An MS-compatible chaotropic agent may be employed in order to avoid a desalting step in the process. The MS-compatible compatible chaotropic agent may be TFE. In some embodiments, the chaotropic agent is a C₂₋₆ organic alcohol, wherein the C₂₋₆ may be a straight or branched-chain alkyl or aryl group, optionally comprising one or more halo substituents. The chaotropic agent may be TFE. In some embodiments, the chaotroptic agent is not guanidine hydrochloride (Gdn HCl), guanidinium thiocyanate, urea, or thiourea. In some embodiments, the chaotropic agent is not a guanidinium salt. In some embodiments, the chaotropic agent is not urea or thiourea. In some embodiments, the chaotroptic agent is not a chaotropic salt. In some embodiments, the chaotropic agent may be immobilized on an eMag bead according to the disclosure. The denaturing step may be performed at a temperature in a range of from about 4° C. to about 60° C., from about 15° C. to about 55° C., from about 20° C. to about 30° C., or about 23° C. The denaturing step may be performed within a range of over a period of time within a range of about 2 min to about 60 min, about 5 min to about 50 min, or about 15 min to about 30 min. The denaturing step may be performed sequentially, overlapping with, or simultaneously with the reducing step.

A “reducing agent” is used to reduce disulfide bonds in the protein analyte, i.e., the cystines, into cysteine thiol moieties. The reducing agent may be any appropriate reducing agent. The reducing agent may be dithiothreitol (DTT), dithioerythritol (DTE), tris(2-carboxyethyl) phosphine hydrochloride (TCEP), beta-mercaptoethanol (OME), or L-glutathione (GSH). The reducing agent may be DTT. The reducing step may be performed at a temperature in a range of from 4° C. to about 60° C., from 15° C. to about 55° C., from about 40° C. to about 50° C., or about 45° C. The reducing step may be performed over a period of time within a range of about 2 min to about 60 min, about 5 min to about 50 min, or about 15 min to about 30 min.

The “alkylating agent” is used to alkylate the free cysteine thiols in the reduced protein analyte. The alkylating agent reacts with cysteine residues in proteins. It is used to modify —SH groups to prevent reformation of disulfide bonds after reduction of cystine residues to cysteine residues. The alkylating agent may be any appropriate alkylating agent. The alkylating agent may be 2-iodoacetamide (IAM), iodoacetic acid (IAC), chloroacetamide (CAA). Alkylating agents may be purchased commercially from, for example, Sigma-Aldrich, or Thermo Fisher. In some embodiments, the alkylating agent is IAM. Alkylation with IAM after cysteine reduction results in covalent addition of a carbamidomethyl group and prevents formation of disulfide bonds. Alkylation of a cysteine residue with 2-iodoacetamide (IAM), chloroacetamide (CAA) or iodacetic acid (IAC) increases the mass of the peptide by, e.g., 57.046, 57.021 or 58.005 Daltons, respectively. This increase in mass may be measured using a mass spectrometer. The alkylating step may be performed at a temperature in a range of from about 4° C. to about 60° C., from about 15° C. to about 55° C., from about 20° C. to about 30° C., or about 23° C. The alkylating step may be performed over a period of time within a range of from about 2 min to about 60 min, about 5 min to about 50 min, or about 15 min to about 35 min, or about 20 min to about 30 min.

The “endopeptidase enzyme” is an enzyme capable of causing proteolytic cleavage within a polypeptide chain rather than at the terminal amino acids located at either end of the polypeptide. For example, the endopeptidase enzyme may be a trypsin, chymotrypsin, LysC, LysN, AspN, GluC, ArgC. In some specific examples, the endopeptidase enzyme may be a trypsin. Suitable endopeptidases are commercially available, for example, from Sigma-Aldrich (Saint Louis, Mo.; Millipore Sigma, Burlington, Mass., subsidiary of Merck KGaA), Promega Corporation, Madison, Wis., or Pierce Biotechnology, Rockford, Ill./Thermo Fisher Scientific Inc.

The endopeptidase enzyme may be a trypsin. Trypsin (EC 3.4.21.4) is a serine protease (serine endopeptidase) enzyme found in the digestive tract of many vertebrates where it hydrolyzes proteins. Trypsin cleaves peptide chains primarily at the carboxyl side of lysine or arginine residues. Trypsin has a pH optima for enzymatic activity of about pH 8.0. The trypsin may be alpha-trypsin or beta-trypsin. Preferential cleavage: Arg-/-Xaa or Lys-/-Xaa. The optimal temperature for activity may vary depending on the type of trypsin. The trypsin may be an isolated trypsin or a synthetic trypsin. The trypsin may be a bovine trypsin or a porcine trypsin. The bovine trypsin may be isolated from bovine pancreas. The bovine trypsin may be recombinant bovine trypsin. The porcine trypsin may be isolated from porcine pancreas. The porcine trypsin may be recombinant porcine trypsin. Trypsin preparations may contain some contaminating chymotrypsin and this may be treated with TPCK (L-1-Tosylamide-2-phenylethyl chloromethyl ketone) to inhibit chymotrypic activity. Free trypsin is commercially available, for example, from Sigma-Aldrich (Saint Louis, Mo.; Millipore Sigma, Burlington, Mass., subsidiary of Merck KGaA) or Promega (Madison, Wis.).

The endopeptidase enzyme may be a chymotrypsin enzyme. Chymotrypsin (EC 3.4.21.1) is a serine protease that cleaves peptide bonds selectively on the carboxyl terminal side of large hydrophobic amino acids such as tryptophan, tyrosine, phenylalanine, and methionine. Chymotrypsin is commercially available, for example, from Promega Corp., Madison, Wis. (chymotrypsin, bovine pancreas).

The endopeptidase may be LysC protease, also known as Lys-C. LysC is a serine protease that hydrolyzes proteins specifically at the carboxyl side of lysine residues. LysC is commercially available form, for example, Promega Corp., Madison, Wis. LysC may be native isolated enzyme or recombinant LysC.

The endopeptidase may be LysN protease, also known as Lys-N. LysN protease is a zinc metalloprotease that may be derived from, for example, Grifolafrondosa. LysN cleaves proteins on the amino-terminal side of lysine residues. LysN protease is commercially available from, for example, ThermoFisher Scientific, Inc.

The endopeptidase may be AspN protease, also known as Asp-N. AspN protease is a zinc metalloprotease derived from Pseudomonas fragi, that cleaves proteins on the amino side of aspartate and cysteic acid residues that result from oxidation of cysteine residues. AsN protease is commercially available from, for example, ThermoFisher Scientific, Inc.

The endopeptidase may be GluC protease, also known as Glu-C. GluC hydrolyzes proteins specifically at the carboxyl side of glutamic acid residues. GluC is a serine protease isolated from Staphylococcus aureus. GluC is commercially available from, for example, ThermoFisher Scientific, Inc.

The endopeptidase may be ArgC protease, also known as Arg-C. ArgC is an endopeptidase that cleaves on the C-terminus or arginine residues, and lysine residues. ArgC may be isolated from Clostridium histolyticum. ArgC is commercially available from, for example Promega Corp., Madison, Wis.

The method comprises enzymatically digesting the pretreated protein analyte to provide a plurality of peptides. The protein analyte may be, for example, a monoclonal antibody, an isolated immunoglobulin, a recombinant protein, an isolated protein, or a combination of different proteins.

In some examples, the enzyme: protein analyte ratio may be in a range or from about 1:5 to about 1:150, from about 1:10 to about 1:100, from about 1:12 to about 1:50, or from about 1:12 to about 1:40, from about 1:20 to about 1:30, or about 1:25. The digesting may comprise incubating the denatured protein analyte with the immobilized enzyme or free enzyme at a temperature of about 32 to 40° C., about 36 to about 38° C., or about 37° C. for a period of time. The period of time may be in a range of from about 5 min to about 75 min, about 10 min to about 60 min, or about 20 min to about 45 min, or about 30 min.

Immobilized trypsin may also be employed. Trypsin immobilized on magnetic particles may be purchased commercially, or may be prepared by any appropriate technique known in the art. The trypsin may be immobilized on a magnetic particle, such as a magnetic bead.

In specific examples, the magnetic bead or magnetic particle suitable for electromagnetic mixing may have a super high magnetic response, for example, having a Bmax of in a range of about 50-250 emu/g, 80-120 emu/g, or about 89 emu/g. This is compared to many commercially available magnetic beads having <40 emu/g. The magnetic bead may have a high surface area of >10 m²/g. The magnetic bead may have a diameter of from about 10 nm to about 200 nm, about 50 nm to about 150 nm, or about 100 nm. The magnetic particle or magnetic bead may be surface functionalized with a carboxyl, amino, hydroxyl, silica, streptavidin, or endopeptidase enzyme moiety. The magnetite particle or magnetic bead may have a magnetite core. The magnetite particle or magnetic bead may have a magnetite core coated with a silica shield layer. The silica shield layer may be attached to a silane linker. The silane linker may be attached to a polymer. The polymer may be surface functionalized with a carboxyl, amino, hydroxyl, silica, streptavidin, endopeptidase enzyme surface functionality. In a specific embodiment, the magnetic bead or magnetic particle has a carboxyl surface functionality. In another specific embodiment, the magnetic bead or magnetic particle has a trypsin surface functionality.

Buffers

Any appropriate buffer may be used for sample dilution and/or endopeptidase digestion, e.g., trypsin digestion. The buffer may have a pH in a range of from about pH 7 to about pH 9. For example, the buffer may be an ammonium bicarbonate buffer, triethylammonium bicarbonate (TEAB) buffer, ammonium acetate buffer, or TRIS-HCl buffer. For example, 0.1 M ammonium bicarbonate, 50 mM ammonium bicarbonate (pH 7.8), or 50 mM Tris-HCl (pH 8) or may be employed. In some embodiments, the buffer does not include urea.

Systems

Magnetic beads have been widely used for sample preparations. However, the magnetic property of particles is typically utilized only during the buffer exchange (supernatant removal) process. The sample mixing with surface-functionalized beads is still typically achieved by mechanical agitation (e.g. shaking, pipette mixing). With these types of traditional methods, magnetic particles may aggregate and cluster in discrete areas close to the walls of the container, greatly reducing mixing efficiency.

The present disclosure provides an improved protein digestion process comprising mixing a protein analyte with a magnetic particle in a changing magnetic field. The changing magnetic field may utilize oscillating electromagnetic fields to fully disaggregate magnetic beads, allowing an optimal exposure and enhanced mixing with surrounding liquid medium. Therefore, the reaction kinetics can be significantly improved.

The process employs an electromagnetic mixer for highly efficient, automated sample preparation. Suitable electromagnetic mixer assemblies for processing fluids are described in WO 2017093896, US 2018-0369831, US 2020-0011773, and WO 2020016854, Arnold et al., and U.S. Ser. No. 10/656,147, Campbell et al., each of which is incorporated by reference herein in its entirety.

In various aspects, a fluid processing system may include a magnetic assembly that includes a plurality of magnetic structures configured to generate a magnetic field gradient within a fluid container. The magnetic structures may be formed as a plurality of electromagnets configured to be individually actuated by a controller. Each of the electromagnets may generate a magnetic field within the fluid container. The electromagnets may be differentially actuated to create a magnetic field gradient within the fluid container to agitate, mix, or otherwise influence magnetic particles disposed within the fluid container. Activation of the electromagnets of an electromagnetic structure may generate a magnetic field gradient that influences magnetic particles in an x-y direction. In addition, activation of the electromagnets of a plurality of electromagnetic structures may generate magnetic field gradients that influences magnetic particles in an x-y direction and z-direction.

The system may comprise a multiwell electromagnetic mixer that is actuated by an array of star-shaped electromagnets, so that each sample well (e.g., from a commercial 24-well plate or 96-well plate) can be placed within the hole formed by four electromagnets. Each electromagnet is used for the field control of, e.g., four sample wells. By applying AC with different phase delays to various electromagnets in the array, all wells can be activated simultaneously, creating a homogenous suspension of magnetic particles in each well, reducing diffusion distances to a minimum and improving reaction kinetics. During any buffer exchange process, a steady state magnetic field (DC) may be used to trap beads at corners of the sample well. The system also may include a temperature control module, containing a hot plate placed above the sample plate, several temperature sensors, and a cooling fan.

The special design of magnetic lenses may allow the electromagnetic mixer to be compatible with most commercial multi-well, e.g., 24-well or 96-well plates. The sample temperature may be precisely controlled by the structure of hotplate, cooling fans, and temperature sensors. It takes less than 5 min to heat up the system from room temperature to 37° C. (and another 5 min to 70° C. if necessary), and the sample temperature can be maintained within the ±1° C. range. Tryptic digestion of Herceptin was evaluated to test this electromagnetic mixer. The mixture of Herceptin (trastuzumab; 10 mg/mL) and trypsin (1 mg/mL) were either incubated using vortex mixing (37° C.) or electromagnetically mixed in the current device (37° C.).

The effective mixing for a high-volume sample (>1 mL) is challenging by mechanical agitation, since the classical 2-D shaking cannot bring particles to the top portion of the solution, and the extensive vertical shaking would cause the problem of solution splash and droplets attaching to the vial cap/cover. The effective and homogeneous 3-D beads mixing for a high sample volume can be achieved by a two-layer electromagnets structure.

For a broad assay coverage, the system is designed to be compatible with both high magnetic response particles (e.g., ferrimagnetic beads), and low response particles (e.g. superparamagnetic beads) by tuning the AC waveforms applying to the electromagnets set. The power consumption even for a sample containing low-response superparamagnetic beads is less than 5 W.

The AC frequency of electromagnetic field may be 300 Hz, and the field strength may be designed as 31 mT p-p (adjustable through the user interface). Different signal phases controlling each electromagnet may be employed to achieve the optimal suspension of magnetic particles. The system may be extended from a single well controlled by 4 electromagnets up to 96 wells controlled by 117 electromagnets with each electromagnet shared by four wells.

Several formats of electromagnetic mixers may be used for the fast and automated sample preparation. The use of AC driven oscillating magnetic fields allows formation of a homogenous “dynamic fog” suspension of magnetic beads, allowing an optimal exposure and enhanced mixing with surrounding liquid medium. The reaction kinetics can be significantly improved using these systems. The design of the “star-shaped” magnetic lenses allows the system compatible with commercial 96-well plates, and with the magnetic particles. The two-layer electromagnets structure may be employed for efficient 3-D mixing for high-volume samples up to 2 mL. The system may comprise an integrated temperature control module. This system may be employed for ultra-fast sample preparation device for the high-throughput analysis.

For samples with a larger volume (e.g. >0.5 mL), a two-layer electromagnets structure may be used to incorporate the active mixing in the z-direction in addition to the x-y mixing. Using this configuration, the 3-D homogeneous particle distribution is successfully observed for samples up to 2 mL.

Significantly improved reaction kinetics is observed with the system comprising the magnetic mixer. Using a model digestion with BSA, a 5-min digestion time is enough to achieve a higher yield than the 2-h digestion with a thermo-shaker. In addition, no significant surface adsorption was observed by adding the magnetic particles in the reaction system.

Processes

In traditional solution digestion, the protein analytes are denatured with strong chaotropic agents such as urea or thiourea. This step is either followed by or combined with disulfide reduction using a reducing agent such as tris (2-carboxyethyl) phosphine (TCEP) or dithiothreitol (DTT). The free sulfhydryl groups on the cysteine residues are then alkylated with alkylating reagents such as iodoacetamide or iodoacetic acid to irreversibly prevent the free sulfhydryls from reforming disulfide bonds. The denatured, reduced and alkylated proteins are then digested by endopeptidase, (e.g., trypsin, chymotrypsin, Glu-C and Lys-C), which hydrolytically break peptide bonds to fragment proteins into peptides. The last two steps of the classic method can be very time-consuming and may require 6 to 20 hours or more to complete a sample preparation.

A method of characterizing a protein analyte is provided comprising pretreating and enzymatically digesting the protein analyte to provide a plurality of peptides. The plurality of peptides may be analyzed by a method comprising mass spectrometry (MS). The plurality of peptides may be separated and analyzed by, for example, liquid chromatography and mass spectrometry (LC-MS or LC-MS/MS). The pretreating may comprise mixing a protein analyte and a chaotropic agent in an aqueous solution with a magnetic particle in a changing magnetic field (to provide a denatured protein solution); adding a reducing agent (to reduce cysteine disulfide bonds) to provide a reduced, denatured protein analyte; and exposing the denatured protein analyte to an alkylating agent (to alkylate the cysteine thiol —SH groups) to provide an alkylated protein analyte. The method may include quenching excess alkylating agent by adding a reducing agent to the alkylated protein analyte. The digesting may comprise mixing the alkylated protein analyte with an endopeptidase enzyme to provide the plurality of peptides.

The plurality of peptides may be separated using a chromatographic technique. The peptides may be analyzed comprising a mass spectrometry technique. The peptides may be separated and analyzed using LC-MS or LC-MS/MS. The protein analyte; reduced, denatured protein analyte; alkylated protein analyte; and/or peptides may be separated and characterized using LC-MS or LC-MS/MS, for example, prior to, during, or after the process, respectively.

The protein analyte may be pretreated comprising electromagnetic mixing with eMBs in a changing magnetic field in order to efficiently denature, reduce and alkylate the protein analyte. The denatured, reduced, and alkylated protein analyte may be mixed with a free trypsin at a pre-selected ratio, e.g., a 25:1 protein analyte:trypsin, (w/w) ratio or with a trypsin immobilized on an eMag bead according to the disclosure. Ty-eMBs at a digestion temperature set at 37° C. for magnetic mixer. After incubation, 2% (v/v) formic acid may be added to the solution, and the samples may be analyzed by LC-MS/MS.

The inventive protein digestion process exhibited SQ %>95%, artifact<5%, and PIMs<10%. The inventive protein digestion processes exhibited reproducibility, CV %<15%. Therefore, the process was at least comparable to prior art “in solution” process.

In some embodiments, the protein pretreatment comprising denaturation, reduction, alkylation, and quench may be completed in <45 min. The protein digestion process may be completed in <60 min, <45 min, or <30 min. A particular advantage of the inventive process is that no vial or tube movement is required thoughout pretreatment and digestion process. The bead suspension is stable over at least 3 min for dispensing purposes. Therefore, the inventive process was simpler and required a shorter time to completion than the prior art “in solution” process.

Kits

The disclosure provides kits suitable for performing the methods provided herein. For example, a kit is provided for preparing a protein analyte for characterization, the kit comprising a first container comprising first magnetic beads; a chaotropic agent; a reducing agent; an alkylating agent; and an endopeptidase enzyme. The endopeptidase enzyme may be a free endopeptidase enzyme or an immobilized endopeptidase enzyme. The immobilized endopeptidase enzyme may be immobilized on second magnetic beads according to the disclosure. The first magnetic beads may have a high magnetic response in a range of from about 50-250 emu/g, 80-120 emu/g, or about 89 emu/g. The first magnetic beads may have a surface functionality selected from carboxyl, amino, hydroxyl, or silica groups. The first magnetic beads do not comprise an immobilized enzyme. The system of the disclosure may comprise an electromagnetic mixer comprising a plurality of electromagnets capable of generating an AC driven oscillating magnetic field and a kit according to the disclosure.

### EXAMPLES

### Example 1. Comparative Prior Art “in Solution” Digestion Using Promega Trypsin (Amgen Method)

This example illustrates a comparative prior art benchmark “in solution” tryptic digestion of a protein analyte monoclonal antibody. The workflow of the prior art in solution tryptic digestion is shown in FIG. 2 (upper panel). Herceptin (Trastuzumab) is a humanized IgG₁ kappa monoclonal antibody that selectively binds to extracellular domain of the human epidermal growth factor receptor 2 protein, HER2. Trastuzumab may be produced by recombinant DNA technology in a mammalian cell (Chinese Hamster Ovary) culture.

0.1 mL 10 mg/mL Herceptin (Trastuzumab) stock solution was diluted to 1 mg/mL in 7.5 M guanidine hydrochloride (Gdn HCl, Sigma) and 0.25 M Tris buffer (pH 7.5). 6 μL of 0.5 M Dithiothreitol (DTT, Thermo Fisher) was added into the mixture followed by 30 min of vortex at 1000 RPM at room temperature. 14 μL of newly prepared 0.5 M 2-Iodoacetamide (IAM, Thermo Fisher) was added into the mixture and incubated on vortex in dark for 15 min at room temperature. 8 μL of 0.5 M DTT was added into the mixture to destroy the access IAM for 30 min on vortex. In a desalting step, the buffer was exchanged to 0.1 M ammonium bicarbonate (Fluka) using Vivaspin 500 centrifugal concentrator (MWCO 10 kDa, Sigma) using Spectrafuge 16 M microcentrifuge. Lyophilized trypsin (Promega, sequence grade or gold grade porcine trypsin) was dissolved in suspension buffer to a final concentration of 1 mg/mL. Trypsin was added into the prepared mAb solution at 1:25 ratio and incubated at 37° C. on vortex for 30 min. The final digest was quenched with the addition of formic acid (FA, Sigma) to 0.2% in the solution. The digested sample was analyzed using LC-MS/MS.

### Example 2. Magnetic Bead Digestion Using Bovine Trypsin Coated eMag Beads or Free Bovine Trypsin

This example illustrates an inventive eMag digestion process of a monoclonal antibody first using non-enzymatic magnetic beads solely as micro-stirring bars for efficient denaturation, reduction, alkylation, and quench, followed by digestion using either bovine trypsin coated eMagBeads or free bovine trypsin (Sigma T8802, TPCK treated trypsin from bovine pancreas).

10 μL 5% magnetic beads (carboxylated beads, used only as micro-stirring bars for mixing) was put into a tube and water was removed. B-500-1-BW utilizes eMagBeads from Lodestone, functionalized with carboxyls, which are used only as micro stirring bars for mixing during denaturation/reduction/alkylation/quench steps. 10 μL of a 10 mg/mL Herceptin stock solution was added into the tube and then mixed with 10 μL trifluoroethanol (TFE, Sigma). 2 μL of 0.25 M DTT was added into the mixture and mixed on the e-mixer for 45 min at ˜45° C. 2 μL of 0.5 M IAM was added into the mixture and mixed in dark on the e-mixer for 30 min at room temperature. Excess IAM was quenched with the addition of 2 μL of 0.25 M DTT and incubation for 45 min at room temperature. The solution was diluted by adding 120 μL water with 7.5 mg Ty-eMB-06 (bovine trypsin coated eMagBeads) and 60 μL 0.1 M ammonium bicarbonate. Then digested for 30 min on the e-mixer at 37° C. Alternatively, when magnetic beads are only being used as stir bar, instead of adding trypsin coated beads, free trypsin is added at this step at a 1:25 enzyme/Mab ratio. After digestion, the magnetic beads were removed and 4 μL 10% FA was added into the solution to quench the digestion. The digested sample was analyzed using LC-MS/MS.

### Example 3. Magnetic Bead Digestion Using Recombinant Porcine Trypsin Coated eMag Beads

This example illustrates an inventive eMag digestion process of a monoclonal antibody first using non-enzymatic magnetic beads solely as micro-stirring bars for efficient denaturation, reduction, alkylation, and quench, followed by digestion using recombinant porcine trypsin coated eMag Beads.

10 μL 5% magnetic beads (carboxylated beads, used only as micro-stirring bars for mixing) was put into a tube and water was removed. 10 μL of a 10 mg/mL Herceptin stock solution was added into the tube and then mixed with 10 μL trifluoroethanol. 2 μL of 0.25 M DTT was added into the mixture and mixed on the e-mixer for 45 min at −45° C. 2 μL of 0.5 M IAM was added into the mixture and mixed in dark on the e-mixer for 30 min at room temperature. Excess IAM was quenched with the addition of 2 μL of 0.25 M DTT and incubation for 45 min at room temperature. The solution was diluted by adding 120 μL water with 7.5 mg Ty-eMB-10 (recombinant porcine trypsin coated eMagBeads) and 60 μL 0.1 M ammonium bicarbonate. Then digested for 30 min on the e-mixer at 37° C. After digestion, the magnetic beads were removed and 4 μL 10% FA was added into the solution to quench the digestion. It was found that the time for sample preparation should be reducible. The digested sample was analyzed using LC-MS/MS.

### Example 4. LC-MS/MS Conditions

An Agilent 1290 HPLC system was employed with a Phenomenex Kinetex 2.6 um Polar C18 100 A, 150×2.1 mm column at 50° C. with a gradient of eluent A water+0.1% formic acid and eluent B acetonitrile+0.1% formic acid was used. The injection volume was set to 5 μL. A SCIEX X500B was used for the analysis of signature peptides and mAb.

### Example 5. UV Analysis and LC-MS/MS Peptide Mapping of Herceptin

The inventive pre-treatment and enzymatic digestion processes according to examples 2 and 3, and comparative prior art “in solution” process of example 1 were each run using protein analyte Herceptin (Trastuzumab). The inventive processes each included pretreating the protein analyte in the presence of first magnetic beads in a changing magnetic field to provide a pretreated protein analyte; and enzymatically digesting the pretreated protein analytes to provide a plurality of peptides.

Briefly, the inventive processes of FIG. according to examples 2 and 3 each utilized chaotropic agent TFE, reducing agent DTT, alkylating agent IAM, quench using added DTT, and enzymatic digestion in ammonium bicarbonate buffer using either immobilized trypsin (bovine trypsin coated eMagbeads) or free trypsin (Promega trypsin), and final digest was quenched with formic acid.

Briefly, the comparative prior art “in solution” process of example 1 as illustrated in FIG. 2, upper panel, utilized chaotropic salt guanidine hydrochloride, reducing agent DTT and mixing by vortex, alkylating using IAM, buffer exchange into ammonium bicarbonate using Vivaspin 500 centrifugal concentrator, and free lyophilized trypsin (Promega, sequence grade or gold grade porcine trypsin), and the final digest was quenched using formic acid.

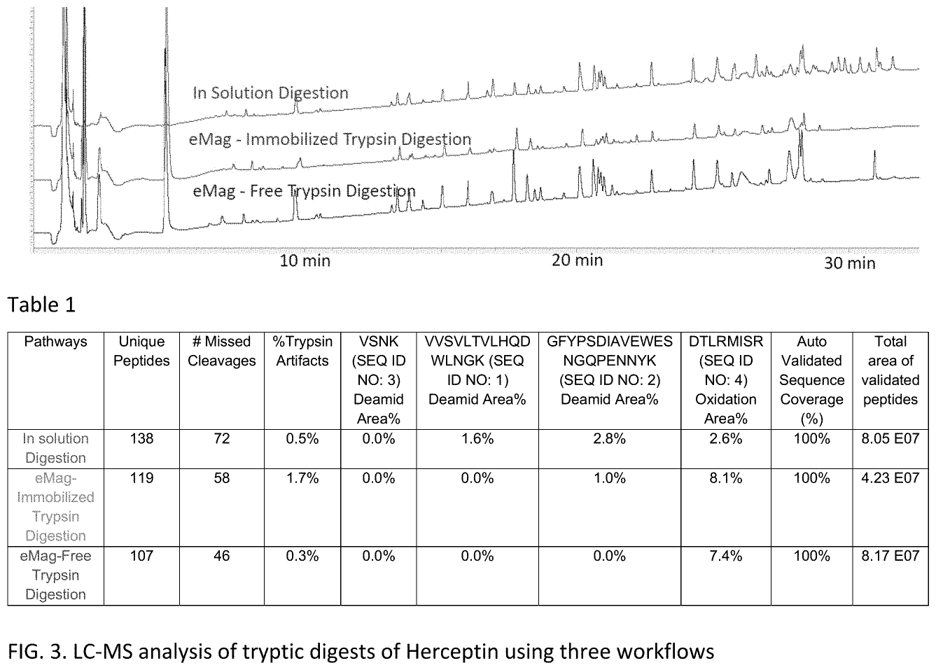

The digested samples from inventive and comparative prior art “in solution” processes were analyzed using LC-MS/MS. FIG. 3 shows results of peptide mapping of monoclonal antibody Herceptin and ultraviolet (UV) spectroscopy and high-resolution mass spectrometry (MS) employing three different processing methods: (i) prior art prior art “in solution” “in solution” trypsin (ii) inventive eMag-Immobilized trypsin, and (iii) inventive eMag-free trypsin.

Prior art “in solution” tryptic process exhibited a higher number of unique peptides (138 unique peptides) than either inventive eMag-immobilized trypsin process (119 unique peptides) or eMag-free trypsin process (107 unique peptides), as shown in FIG. 3, Table 1.

In general, both inventive eMag process pathways, eMag-immobilized trypsin process and eMag-free trypsin process, exhibited fewer process-induced modifications than comparative prior art “in solution” free trypsin process, as shown in Table 1.

Comparative prior art “in solution” free trypsin process exhibited a higher number of undesirable missed cleavages than either inventive eMag-immobilized trypsin process (58 missed cleavages) or eMag-free trypsin process (46 missed cleavages), as shown in FIG. 3, Table 1.

Comparative prior art “in solution” tryptic digest process exhibited a relatively high 1.6% area percent of undesirable deamidation peptide VVSVLTVLHQDWLNGK (SEQ ID NO: 1), whereas inventive eMag-immobilized trypsin process and inventive eMag-free trypsin process did not exhibit a measurable area % for this deamidation peptide, as shown in FIG. 3, Table 1.

Comparative prior art “in solution” trypsin process exhibited a relatively high area percent of 2.8% area of undesirable deamidation peptide GFYPSDIAVEWESNGQPENNYK (SEQ ID NO: 2). In contrast, inventive eMag-immobilized trypsin process exhibited 1.0%, and inventive eMag-free trypsin process did not exhibit a measurable area % of this deamidation peptide, as shown in FIG. 3, Table 1.

Although comparative prior art “in solution” free trypsin process exhibited lower methionine oxidation of DTLMISR (SEQ ID NO: 4) (2.6%), it was found that for both inventive eMag pathways, the 6-10% oxidation of DTLMISR (SEQ ID NO: 4) could be reduced to <5% after T tuning (temperature tuning) for pretreatment.

Reproducibility of inventive eMag tryptic digests and prior art in solution tryptic digests of Herceptin were evaluated with analysis by LC-MS/MS.

In general, inventive eMag digestion methods employing eMag-immobilized trypsin and eMag-free trypsin exhibited fewer unique peptides, fewer missed cleavages, and lower deamidation area % for VVSVLTVLHQDWLNGK (SEQ ID NO: 1) and GFYPSDIAVEWESNGQPENNYK (SEQ ID NO: 2) peptides, when compared to prior art “in solution” free trypsin method.

Both inventive workflow processes eMag-immobilized trypsin and eMag-Free trypsin are capable of exhibiting SQ %>95%, artifact<5%, deamidation<5%, and digestion time<60 min. Each of prior art “in solution”, eMag immobilized and eMag-Free trypsin processes exhibited 0% deamidation for VSNK (SEQ ID NO: 3). * For both inventive eMag pathways, the 6-10% oxidation of DTLMISR (SEQ ID NO: 4) could be reduced to <5% after T tuning (temperature tuning) for pretreatment.

1. A method of preparing a protein analyte for characterization comprising
pretreating a protein analyte in the presence of first magnetic beads in a changing magnetic field to provide a pretreated protein analyte; and
enzymatically digesting the pretreated protein analyte to provide a plurality of peptides.

2. The method of claim 1, wherein the characterization comprises
analyzing the plurality of peptides comprising LC-MS/MS.

3. The method of claim 1, wherein the pretreating comprises
mixing the protein analyte and a chaotropic agent in an aqueous buffer with the first magnetic beads in the changing magnetic field to provide a denatured protein solution.

4. The method of claim 3, wherein the mixing comprises
adding a reducing agent to the denatured protein solution to provide a reduced, denatured protein analyte.

5. The method of claim 4, wherein the mixing comprises
exposing the denatured protein analyte to an alkylating agent to provide an alkylated protein analyte.

6. The method of claim 5, wherein the mixing comprises
quenching excess alkylating agent by adding a reducing agent to the alkylated protein analyte to provide the pretreated protein analyte.

7. The method of claim 1, wherein the digesting comprises
blending the pretreated protein analyte with an endopeptidase enzyme in the presence of the first magnetic beads in a changing magnetic field to provide the plurality of peptides.

8. The method of claim 7, wherein the endopeptidase enzyme is selected from the group consisting of a trypsin, chymotrypsin, LysC, LysN, AspN, GluC, and ArgC.

9. The method of claim 8, wherein the endopeptidase enzyme is a free endopeptidase enzyme or an immobilized endopeptidase enzyme.

10. The method of claim 9, wherein the immobilized endopeptidase enzyme is immobilized on the surface of second magnetic beads.

11. The method of claim 10, wherein the first magnetic beads and the second magnetic beads independently comprise a ferrimagnetic particle, superparamagnetic particle, ferromagnetic particle, paramagnetic particle, or mixtures thereof.

12. The method of claim 11, wherein at least one of the first magnetic beads and the second magnetic beads independently have a high magnetic response having a Bmax in a range of from about 20 emu/g to about 250 emu/g, 40 emu/g to 200 emu/g, 50 emu/g to 150 emu/g, or 80 emu/g to 100 emu/g.

13. The method of claim 11, wherein at least one of the first magnetic beads and the second magnetic beads independently have a high surface area of >5 m²/g.

14. The method of claim 11, wherein at least one of the first magnetic beads and the second magnetic beads independently further comprise a coating, optionally wherein the coating is selected from the group consisting of a silica shield, silane linker, and a polymer coating.

15. The method of claim 11, wherein at least one of the first magnetic beads and the second magnetic beads independently comprise a functional group-coated surface, optionally wherein the functional-group coated surface is selected from the group consisting of carboxyl groups, amino groups, hydroxyl groups, thiol groups, tosyl groups, epoxy groups, alkyl groups, vinyl groups, and aryl groups.

16. The method of claim 11, wherein at least one of the first magnetic beads and the second magnetic beads independently comprise a bioaffinity adsorbent, optionally selected from the group consisting of streptavidin, avidine, neutravidin, captavidin, and biotin.

17. The method of claim 1, wherein the first magnetic beads do not comprise an immobilized endopeptidase enzyme.

18. The method of claim 1, wherein the changing magnetic field is generated in an electromagnetic mixer.

19. The method of claim 18, wherein the electromagnetic mixer comprises a plurality of electromagnets capable of generating an AC driven oscillating magnetic field.

20. The method of claim 1, wherein the protein analyte is selected from the group consisting of a monoclonal antibody, an isolated immunoglobulin, a recombinant protein, an isolated protein, and a complex protein sample; optionally, wherein the complex protein sample is selected from the group consisting of a tissue sample, blood sample, serum sample, and plasma sample.

21. The method of claim 3, wherein the chaotropic agent is an organic chaotropic agent.

22. The method of claim 21, wherein the organic chaotropic agent is selected from the group consisting of trifluoroethanol (TFE), ethanol, 2-propanol, n-butanol, and phenol.

23. The method of claim 3, wherein the chaotropic agent is an immobilized chaotropic agent; optionally wherein the chaotropic agent is immobilized on the first magnetic beads or on third magnetic beads.

24. The method of claim 4, wherein the reducing agent is selected from the group consisting of dithiothreitol (DTT), dithioerythritol (DTE), tris(2-carboxyethyl) phosphine hydrochloride (TCEP), beta-mercaptoethanol (βME), and L-glutathione (GSH).

25. The method of claim 5, wherein the alkylating agent is selected from the group consisting of 2-iodoacetamide (IAM), iodoacetic acid (IAC), and chloroacetamide (CAA).

26. The method of claim 1, wherein the pretreating is completed in a period of time within a range of from 5 to 90 minutes.

27. The method of claim 1, wherein the enzymatically digesting is completed in a period of time from 5 to 60 minutes.

28. The method of claim 1, wherein the pretreating and the enzymatically digesting are performed sequentially or simultaneously.

29. A kit for preparing a protein analyte for characterization according to claim 1 comprising
a first container comprising first magnetic beads;
a chaotropic agent;
a reducing agent;
an alkylating agent; and
an endopeptidase enzyme.

30.-35. (canceled)

36. A system comprising the kit of claim 29, and an electromagnetic mixer comprising a plurality of electromagnets capable of generating an AC driven oscillating magnetic field.


SEQUENCE LISTING (4 sequences)

SEQ ID NO: 1 - protein, 16 aa, synthetic construct
VVSVLTVLHQDWLNGK

SEQ ID NO: 2 - protein, 22 aa, synthetic construct
GFYPSDIAVEWESNGQPENNYK

SEQ ID NO: 3 - protein, 4 aa, synthetic construct
VSNK

SEQ ID NO: 4 - protein, 7 aa, synthetic construct
DTLMISR